(19) United States
(12) Patent Application Publication
(10) Pub. No.: US 2022/0387716 A1
(43) Pub. Date: Dec. 8, 2022

(54) Label for an Injection Device

(71) Applicant: Sanofi, Paris (FR)

(72) Inventor: Michael Helmer, Frankfurt am Main (DE)

(21) Appl. No.: 17/771,288

(22) PCT Filed: Oct. 26, 2020
(86) PCT No.: PCT/EP2020/080004

§ 371 Date: Apr. 22, 2022

(30) Foreign Application Priority Data
Oct. 28, 2019 (EP) 19306398.9

(51) Int. Cl. A61M 5/31

(52) U.S. Cl. CPC A61M 5/31; A61M 2205/502; A61M 2005/3125; A61M 2205/584; A61M 2005/3126

(57) ABSTRACT

The present disclosure relates to a label for an injection device. The label includes a flexible substrate configured for attachment to a body. The label further includes a first label area on the substrate provided with a first information content, wherein the first information content includes at least one visual sign or character. The label further includes a second label area on the substrate non-overlapping with the first label area, wherein the second label area includes an electronic display configured to display a second information content, The label further includes a processor connected to the electronic display and configured to modify at least the second information content.

### CROSS REFERENCE TO RELATED APPLICATIONS

The present application is the national stage entry of International Patent Application No. PCT/EP2020/080004, filed on Oct. 26, 2020, and claims priority to Application No. EP 19306398.9, filed on Oct. 28, 2019, the disclosures of which are incorporated herein by reference.

### TECHNICAL FIELD

The present disclosure relates to the field of labels for injection devices. In particular, the present disclosure relates to a multifunctional label for an injection device that is configured to change its visual appearance. In another aspect the label is configured to remind and/or to assist a user to conduct an injection procedure when making use of the injection device. In another aspect the disclosure relates to an adapter for attaching a label to an injection device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors have to meet a number of user-specific requirements. For instance, with patient's suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

### SUMMARY

Typically, such devices include a housing or a particular cartridge holder, adapted to receive a cartridge at least partially filled with the medicament to be dispensed. The device further includes a drive mechanism, usually having a displaceable piston rod to operably engage with a bung or piston of the cartridge. By means of the drive mechanism and its piston rod, the bung or piston of the cartridge is displaceable in a distal or dispensing direction and may therefore expel a predefined amount of the medicament via a piercing assembly, e.g. in form of an injection needle, which is to be releasably coupled with a distal end section of the housing of the drug delivery device.

The medicament to be dispensed by the drug delivery device may be provided and contained in a multi-dose cartridge. Such cartridges typically include a vitreous barrel sealed in distal direction by means of a pierceable seal and being further sealed in proximal direction by the bung. With reusable drug delivery devices an empty cartridge is replaceable by a new one. In contrast to that, drug delivery devices of disposable type are to be entirely discarded when the medicament in the cartridge has been dispensed or used-up.

With some drug delivery devices, such as pen-type injection devices a user has to set a dose of equal or variable size by rotating a dose dial in a clockwise or dose-incrementing direction relative to a body or housing of the injection device. For injecting and expelling of a dose of a liquid medicament the user has to depress a trigger or dose button in a distal direction and hence towards the body or housing of the injection device. Typically, the user uses his thumb for exerting a distally directed pressure onto the dose button, which may be located at a proximal end of the dose dial and the dose dial sleeve, while holding the housing of the injection device with the remaining fingers of the same hand.

For mechanically implemented injection devices it is desirable to enable a precise, reliable and quasi-automated supervision and/or collection of injection-related data during use of the injection device. Moreover there is a rising demand of user assistance for the proper and regular handling of such drug delivery or injection devices. For a successful therapy a well-defined, e.g. user specific amount of a medicament, i.e. a dose of the medicament, must be administered in accordance to a given prescription schedule, e.g. on a regular temporal basis. In some instances a patient may have forgotten or may not be aware if a prescribed dose was recently injected or not. Moreover, the patient may not always be aware about the size of the dose to be set and injected. This is of particular relevance for patients being oblivious and/or for patients being mentally and/or physically infirm at least to a certain degree.

It is therefore a demand, that information about the medicament and/or the proper or intended handling of the injection device are unequivocally and clearly visible on the outside of the injection device. Medicament-related information and/or handling related information should be provided in a durable and persistent way. However, with some scenarios of use some information should be provided dynamically and/or on demand, e.g. in order to assist a user in or during handling or use of the injection device.

It is therefore an aim to provide an improved label for an injection device, which on the one hand provides durable and persistent information about the medicament and which on the other hand provides supplemental on-demand information regarding a proper and/or intended use of the injection device. The implementation and understanding of the label should be easy and intuitive. It should be manufacturable at moderate or low costs and should enable retrofitting existing injection devices with the improved label. The label should provide a space saving design and should conform to the geometry of a housing of an injection device.

In one aspect the disclosure relates to a label for an injection device. The label includes a flexible substrate. The flexible substrate is configured for attachment to a body. For instance, the flexible substrate is configured for attachment to a body of a housing of the injection device. The label includes a first label area on the substrate. The first label area is provided with a first information content. The first information content includes at least one visual sign or character.

The label further includes a second label area on the substrate. The second label area is non-overlapping with the first label area. The second label area includes an electronic display. The electronic display is configured to display a second information content.

Typically, the first label area and the second label area are arranged on the substrate in a non-overlapping manner. Hence, the first label area and the second label area are arranged next to each other on the substrate. Moreover, at least one of the first label area and the second label area may be at least partially surrounded by the other one of the first label area and the second label area. With some examples the geometric dimensions and shapes of the first label area and the second label areas may be somewhat identical or similar. With some other examples, the geometric dimension and the shape of the first label area differs from the size and geometric shape of the second label area.

The label further includes a processor connected to the electronic display. The processor is configured to modify at least the second information content to be provided by the electronic display of the second label area. In this way, the label provides a twofold function. Typically, the first label area is configured to persistently or to non-alterably provide the first information content. Typically, the first information content is indicative of the type of the medicament located or stored in the injection device. The second label area is reconfigurable. Hence, the second information content may change on demand. Typically, the second label area is configured to vary and to modify the second information content either deterministically, on demand or in dependency of at least one environmental or ambient condition.

Typically, the second information content to be provided by the electronic display in the second label area may provide information suitable to assist a user in a proper or intended handling of the injection device and/or use of the medicament located therein. With typical examples the second information content is indicative of at least one: of a point of time at which a next use of the device or an injection is due, an amount or a size of the dose to be set and/or to be dispensed by the injection device, a current state or configuration of the injection device or user instructions being indicative of the correct handling of the injection device for the purpose of setting and/or expelling or dispensing a dose of the medicament.

Typically, the first label area and the second label area are of different types. The first label area is particularly configured to persistently and/or to non-alterably provide information with regards to the type of medicament and/or properties of the medicament. The second label area is particularly configured to provide dynamic information regarding the correct use and handling of the medicament and/or of the injection device. At least the second label area is configured to provide temporally varying information contents. Hence, the processor connected to the electronic display of the second label area is configured to modify the visual appearance of the second label area and/or to modify the second information content over time.

The label is not limited for attachment to an injection device. The label is generally suitable for attachment to other medical devices, such as an infusion device, an infusion pump, an injection pump, an inhaler, or a portable medical analysis device, such as a blood glucose meter. Each of these devices typically includes a body, e.g. implemented as a component of a device housing. The respective body or housing is configured to engage with or to receive the flexible substrate. Insofar, the label and hence the flexible substrate thereof is attachable to a body of a medical device. The medical device may be implemented as an injection device, e.g. as a handheld injection device, as an infusion device, as a drug delivery pump, as an inhaler or as a portable medical analysis device, such as a blood glucose meter.

Typically, the body is of elongated shape. With some examples the body includes a tubular shaped body. With some examples the body includes a mechanically rigid structure. With some examples the body includes an injection molded plastic component.

According to a further example the first label area includes an electronic display of a first display type. The electronic display of the second label area is an electronic display of a second display type. The first display type and the second display type are different display types. Typically, the electronic display of the first label area is a first electronic display. The electronic display of the second label area is a second electronic display. First and second electronic displays are of different display types. The first electronic display of the first electronic display type is particularly configured to provide the first information content in a persistent and durable, e.g. non-erasable way. The second display and/or the second display type is configured to provide a dynamically changing or a dynamically modifying second information content on the second label area and hence on the second electronic display.

By providing two different types of electronic displays only one and the same substrate and/or one and the same label provides a twofold function. Regulatory provisions typically require that at least a type or name of a medicament is persistently and/or non-erasably visible on the outside of an injection device. With the first electronic display and/or with the first display type this requirement can be fulfilled. Typically, the first display type and/or the first electronic display may persistently provide the first information content even in the absence of an electric energy supply. The first electronic display and/or the first display type may be characterized by a rather low electric power consumption. Typically, the electric power consumption of the first display is less than an electric power consumption of the second display.

The second electronic display and hence the second display type may be configured to provide a dynamically varying second information content. The second information content providing instructions or information regarding use of the injection device may be allowed to disappear in the event that the label should be insufficiently provided with electric energy for driving or operating the second electronic display. Nevertheless and since the first display is of persistent type the absence of electric energy has no detrimental effect on the persistence, durability and/or readability of the first information content provided by the first electronic display in the first label area.

According to another example the first display type is one of an electroluminescent display, an electrophoretic display, a liquid crystal display and a light emitting diode display. Likewise, the second display type is one of an electroluminescent display, an electrophoretic display, a liquid crystal display and a light emitting diode display. First and second display types are different.

Typically, the first display type consumes less electric power compared to the second display type. With some examples the first electronic display and the first display type is also connected to the processor or is provided with a separate processor. In this way, also the first display may be reconfigured and may provide that the first information content varies over time. However, the first information content may be always conform with regulatory provisions. Each available appearance of the first information content fulfills legal and/or regulatory requirements regarding a visual illustration of the medicament located or arranged in the injection device or intended for use with the injection device.

According to a further example the first display type is an electrophoretic display and the second display type is a light emitting diode display. Insofar the first display is an electrophoretic display and the second display is a light emitting diode display. Typically, the second display is an organic light emitting diode display. Both, the first display and the second display may be of flexible type. Both, the first display and the second display may include a flexible sheet display. The first display and/or the second display may include numerous display layers that are flexible. With some examples, the first display and/or the second display include flexible display layers stacked on top of each other. Both, the first and the second display may be arranged on a common flexible substrate.

Implementing the first display as an electrophoretic display enables to provide a persistent and durable first information content on the first label area even if the label should be insufficiently supplied with electric energy. Implementing the second display and hence the second label area as a light emitting diode display provides excellent contrast readability and brightness. Moreover, light emitting diode display can be provided with a comparatively high resolution, such as more than 50 pixels per inch (PPI), more than 70 ppi, more than 100 ppi, more than 120 ppi, more than 140 ppi or even more than 150 PPI.

According to another example the first information content is printed or imprinted on the first label area. With this example the first label area is a void of an electronic display. Rather, the first label area is durably and/or persistently printed with the first information content. Here, medicament related information, such as a medicament name, a medicament type, a manufacturing date or manufacturing LOT is provided in printed or imprinted form on the first label area. Here, the first label area and the second label area, the latter of which provided with the electronic display are provided on one and the same flexible substrate.

Having a printed or imprinted first label area and an electronic display constituting a second label area on one and the same flexible substrate provides the benefit that only one label, i.e. only one common flexible substrate has to be attached to the injection device in order to provide both, persistent and non-erasable medicament-related information to the user and dynamically modifiable information or instructions to a user. Manufacturing and assembly of the injection device may be therefore enhanced and simplified.

According to another example the electronic display of the second label area includes an organic light emitting diode (OLED) display. There, the first information content may be printed or imprinted on the first label area while the second label area is provided with or constituted by the organic light emitting diode display.

With some other examples the label of the injection device may be void of a first label area on the substrate. Here, the flexible substrate may be provided with the organic light emitting diode display connected to and driven or operated by the processor in order to provide the reconfigurable second information content. When the second label area is provided with an organic light emitting diode display the label may be even void of a first label area. An organic light emitting diode display is flexible and/or bendable. It may conform the shape of the flexible substrate when attached and/or wrapped to the body, which may be of tubular shape. The organic light emitting diode display further provides excellent readability, contrast, brightness and resolution.

According to another example the electronic display of the second label area includes a color display configured to visualize at least one of the second information content and an information background in at least two different colors. Hence, the second electronic display is a color display. With further examples, the electronic display of the second label area, hence the second electronic display, is configured to modify the brightness of at least one of the second information content and the information background.

With further examples the processor connected to the second electronic display may be configured to switch of or to deactivate the second electronic display. In this way, an electric power consumption of the second electronic display can be reduced to a minimum. The processor may be further configured to wake up or to switch on the second display. In this way, the second information content can be provided selectively, i.e. only in situations in which the second information content should be displayed and provided to a user.

With some examples, the processor includes a power management. The power management may be operable to switch on and/or to switch off the electronic display, e.g. the second electronic display, either deterministically, e.g. after lapse of a predefined time interval. The power management may be configured to switch on and/or to switch off the respective electronic display on demand, e.g. when a user interacts with the label and/or with the processor, e.g. via an input.

Moreover and with further examples the power management may be configured to switch on and/or switch off the electronic display, e.g. the second electronic display, dependent on varying ambient conditions. For this, the power management may be connected to at least one sensor, such as an ambient sensor configured to detect varying ambient conditions, such as ambient brightness, temperature, humidity, acceleration and/or varying air pressure.

Instead of switching the respective electronic display off the power management may be further operable to dim the electronic display, hence to reduce the brightness of at least one of the information content and the information background. In this way power consumption of the respective electronic display, i.e. at least of the second electronic display can be reduced.

According to another example the label includes at least one of a device sensor and an ambient sensor. The device sensor is configured to determine at least one of a position or orientation of at least one of a dose tracker and a piston rod of the injection device relative to a housing or body of the injection device. The ambient sensor is configured to determine at least one of an ambient brightness, an acceleration, an ambient temperature and a variation of ambient air pressure. Signals generated and obtained from at least one of the device sensor and an ambient sensor are transmitted to the processor of the label. Signals of at least one of the device sensor and the ambient sensor are processable by the processor in order to modify the visual appearance of at least one of the first display and the second display.

Hence, the processor is operable to receive and to process at least one signal of the device sensor and/or of the ambient sensor. The respective sensor signal is indicative of a modification or change of at least one of a position or orientation of at least one of the dose tracker and the piston rod. Moreover or alternatively, the sensor signal can be indicative of a change of the ambient brightness, the acceleration, the temperature or a variation of the ambient air pressure. Accordingly and dependent of the signals(s) received from the ambient sensor and/or from the device sensor the processor will modify the visual appearance of at least one of the first label area and the second label area.

With further examples the processor is operable to modify at least one of the first information content and the second information content depending on an electric signal received from at least one of the device sensor and the ambient sensor. Here, signals obtained from the device sensor, which may be indicative of a momentary state or condition of the injection device, may be processed and may be displayed on the second electronic display. For instance, a size of a dose currently set and/or detected by the device sensor may be visually displayed on the second electronic display and hence in the second label area of the label. In addition or alternative, the second information content may provide user instructions regarding the proper handling of the injection device for setting and/or for dispensing of the dose.

Detection of a particular acceleration by the ambient sensor may be indicative that the injection device is subject to a movement, e.g. when the injection device is gripped or taken by a user. Detection of an acceleration above a predefined threshold may be used to wake up the processor and/or the second electronic display. E.g. upon gripping and/or upon lifting or turning of the injection device the processor may be configured to switch on the second electronic display or to increase the brightness of the second electronic display.

Moreover and according to further example the ambient sensor may be integrated into the power management of the label. If the ambient sensor detects a variation of ambient conditions, such as ambient brightness, ambient temperature, ambient humidity, acceleration, or ambient air pressure the processor may set the label and/or the device sensor into an activated state. Otherwise and if ambient conditions have not changed over time and/or if ambient conditions are not subject to a modification over a time interval exceeding a predefined inactivity time interval the processor and/or the power management may be configured to set the label, e.g. the processor itself and/or the second electronic display into a sleep mode or idle mode. In the sleep mode or idle mode only the ambient sensor may remain active.

Upon detection of a variation of ambient conditions the processor may be operable to activate the device sensor at least for a predefined time interval, i.e. an activation time interval. During the activation time interval signals generated by and/or obtained from the device sensor are processed by the processor in order to monitor and/or to assist setting and/or dispensing of a dose with the injection device.

Signals received from the ambient sensor may be used to wake up the processor and/or at least one of the first and second electronic displays. For instance and when the ambient sensor detects an acceleration above a predefined threshold, which acceleration being for instance indicative that a user grips or flips the injection device when the label is attached to the injection device the appearance of the second label area and/or the appearance of the first label area may be subject to a change. In this way the wake up of the processor may be visually indicated to a user of the device. Moreover, on the basis of electric signals received from the ambient sensor the processor may be configured to modify the first information content of the first label area, e.g. to modify the information content on the first electronic display of the first label area.

Here, upon gripping and/or upon lifting of the injection device a first portion of medicament related information and a second portion of medicament related information may be alternately illustrated and provided on the first electronic display. In this way, the readability of the first information content can be increased. The first information content may be reproduced with enlarged signs or letters. In this way medicament related information can be provided on the first electronic display at a size that would normally exceed the display size. By alternately switching or modifying the first information content on the first electronic display medicament-related information can be split into at least first and second pieces of information that are alternatively displayed on the first electronic display. In this way readability of the medicament related information can be increased and improved.

According to a further example the processor is operable to turn or to flip an orientation of at least one of the first information content and the second information content depending on an electric signal received from the ambient sensor. The ambient sensor may be implemented as an orientation sensor and/or as a tilt sensor. In this way, the orientation of the label and/or of the injection device to which the label is attached to can be determined with regard to the direction of the ambient gravitational field. If a user should grip the injection device and/or if a user should turn or flip the injection device in a direction or orientation in which the first and/or the second information content is generally unreadable the processor is operable to turn or to flip the orientation of at least one of the first information content and the second information content accordingly.

In this way the visual appearance of the first and/or second information content of the first and second label areas, hence the information content on the first and the second electronic displays can be dynamically adapted to the momentary orientation of the label and/or of the injection device. In this way it is guaranteed, that the label, i.e. the first and the second information contents are readable in either available or conceivable orientation of the label.

In another aspect there is provided a label for an injection device. The label includes a flexible substrate configured for attachment to a tubular-shaped body. The label includes a first label area on the substrate. The first label area includes a first electronic display configured to display a first information content on the first label area. The label further includes a processor connected to the first electronic display and configured to modify at least the first information content to be provided on the first electronic display. The label further includes an orientation sensor configured to determine the orientation of the label with respect to the ambient gravitational field.

The processor is configured to modify, in particular to turn or to flip an orientation of the first information content of the first electronic display dependent on signals received from the orientation sensor. This particular label for the injection device may include all features of the label as described above and as will be described below but is equipped with only one electronic display. It may be void of a second label area and/or it may be void of a first information content printed or imprinted of the substrate.

According to another example the device sensor of the label includes an electrode structure on the substrate. The electrode structure is configured to measure at least one of an electric capacitance, an electric field and a magnetic field. Typically, the injection device is equipped with and/or includes a dose tracker the position or orientation of which in relation to the housing or body of the injection device is indicative of a size of a dose currently set or dispensed. The dose tracker may be provided with an electronically, electrically and/or magnetically detectable feature. In this way, the device sensor with its electrode structure is configured and/or operable to determine a position and/or orientation of the dose tracker of the injection device during handling and operation of the injection device for setting and/or dispensing of a dose of the medicament.

The device sensor may be a contactless sensor. The electrode structure may be embedded in the flexible substrate. The electrode structure may be arranged on an upper side of the flexible substrate. A lower side of the flexible substrate may be provided with an adhesive for attaching the flexible substrate and hence the label to the body. The electrode structure for measuring at least one of an electric capacitance, an electric field and a magnetic field enables a contactless determination and/or quantitative measuring of a position or orientation of the dose tracker of the injection device relative to a housing or body of the injection device when the label is attached to the injection device.

According to a further example the electronic display includes at least one electrically conductive grid electrode at least partially spatially overlapping with the electrode structure of the device sensor. The at least one electrically conductive grid electrode provides an electromagnetic shield for the electrode structure of the device sensor. This is of particular advantage when the electrode structure is susceptible to ambient electric or electromagnetic fields. Here, the conductive grid electrode of the electronic display may provide a double function. The conductive grid electrode is operable to drive and/or to operate the first electronic display and/or the second electronic display. At the same time, the electrically conductive grid electrode serves to screen and/or to shield the electrode structure of the device sensor that is typically located underneath.

In a layer stack of the label the electrode structure of the device sensor is provided on an upper side of the flexible substrate. Typically, the at least one electrically conductive grid electrode is arranged on top of the electrode structure of the device sensor. Hence, the electrode structure of the device sensor is located between the flexible substrate and the at least one electrically conductive grid electrode.

With other examples, the electrode structure of the device sensor is located or arranged on a lower side of the flexible substrate. Then, the at least one electrically conductive grid electrode is located on an upper side or outside facing portion of the flexible substrate. In either way, the electrode structure of the device sensor is effectively shielded against ambient electromagnetic or electrostatic perturbations.

The flexible substrate is particularly configured to wrap around at least a portion of the body or housing of the injection device. The body or housing may include a somewhat cylindrical or tubular structure. The flexible substrate may hence conform or customize to a sidewall of the tubular shaped housing of the injection device. The flexible substrate enables a rather universal attachment of the label to a multitude of different injection devices. The label may be universally applicable and/or attachable to different types of injection devices.

The label is also rather thin or flat and can be permanently attached to the housing of the injection device. The injection device with the label attached thereto can be wrapped or packed in a device packaging, e.g. for transportation and/or storage. If at all, the label only has a minor impact on the outer contour and/or geometry of the injection device when attached to the housing of the injection device. The label may further impart a rather attractive design to the injection device.

The flexible substrate may include a flexible plastic foil or a flexible metal foil. The flexible substrate may include a plastic foil e.g. made of polyethylene (PE) or polyethylene terephthalate (PET).

The flexible substrate, the electronic display and hence the entire label may include a total thickness of less than 5 mm, less than 4 mm, less than 3 mm or less than 2 mm. With some examples the label includes a thickness of less than 1 mm or less than 0.5 mm.

Typically, and with numerous examples the label includes an electronic circuit located on the substrate. The electronic circuit is electrically connected to the processor and to the electronic display. The electronic circuit may be directly arranged on the flexible substrate. The electronic display and/or the processor may be integrated into the electronic circuit.

The electronic circuit may include a flexible electronic circuit. Hence, electrically conductive structures of the electronic circuit are bendable or pliable so as to follow a flexible deformation of the substrate, e.g. in the course of attaching the flexible substrate to the housing of the injection device, e.g. through wrapping the label to the tubular shaped housing.

The electronic circuit may be printed on the substrate. The electronic circuit may be printed on the flexible substrate by one or more inks that are composed of carbon-based compounds. Moreover, the electronic circuit may be deposited on the flexible substrate by a solution-based or vapor-based deposition process. A printed electronic circuit on the substrate enables a low cost volume fabrication of the label.

In another example a lower side of the flexible substrate is at least in sections provided with an adhesive. The lower side of the flexible substrate may be provided with an adhesive layer. The entire lower side of the flexible substrate or only portions thereof may be provided with the adhesive. Especially the border regions of the lower side of the flexible substrate are provided with the adhesive in order to enable a permanent attachment of the label to the housing of the injection device. By means of the adhesive on the lower side of the flexible substrate an adhesion-based fixing of the label to a body or housing of the injection device or adapter can be provided. This is rather space saving and can be implemented at moderate or low cost.

According to another example the electronic circuit further includes a battery. The battery is typically connected to the processor and/or to the electronic display. The processor and/or the battery may be printed on the flexible substrate. The processor and/or the battery may also be flexible to a certain degree thus allowing and supporting a deformation of the flexible substrate upon assembly and attachment to the housing of the injection device.

The processor is connected to the electronic display and is also connected to the battery. The processor is driven by electric energy provided by the battery. Typically, both the processor and the battery include or are made of printed electronic components.

With further examples the electronic circuit includes a data storage or memory configured to store at least one of a number of user activities and a point of time of a user activity or of numerous user activities. The electronic circuit and/or the processor may be further equipped with a clock to derive a time indication and in order to enable storage of a point of time of a user activity, e.g. a point of time at which a user touches or depresses the touch sensitive area of the label. If the label is void of a clock the electronic circuit may be simply configured to count a number of such user activities. In this way, a kind of a dosage counter can be provided by the label.

According to another example the electronic circuit includes an antenna for wireless transmission of electronic signals with an external electronic device. The antenna is typically connected to the processor. By means of the antenna the processor is configured to communicate with an external electronic device. The processor may be configured to transmit data to the external electronic device. The processor may be configured to receive data from the external electronic device. The external electronic device may include a handheld external electronic device, such as a smartphone, a smart watch or a tablet computer. The antenna may be further enabled to provide communication with a personal computer or similar computing devices.

The antenna and its interaction with the processor further enables a transfer of data previously stored in the data storage of the electronic circuit to the external electronic device. By means of the antenna the external electronic device may be configured to read out the content of the data storage of the label. Typically, the data storage is integrated into the electronic circuit. With other examples it may be provided separately, hence offset from the electronic circuit. The wireless transmission of electronic signals between the processor and the external electronic device further enables a reconfiguring of the processor and/or of the electronic display.

With the external electronic device and the wireless transmission of electronic signals the external electronic device may be used to reconfigure the label. In this way, the at least one indication may be replaced by a second indication. In addition, the total appearance of the electronic display may be reconfigured in accordance to electronic signals received from the external electronic device. In this way, the at least one electronic display can be individually configured for different usage scenarios and/or for use with different injection devices.

The transmission of data stored in the data storage of the electronic circuit to the external electronic device enables a monitoring and post-dispensing evaluation of user activities that were recorded in the data storage at during time intervals during which the label is disconnected from the external electronic device.

Even though the label as described herein is particularly configured for attachment to an injection device the label may be equally attachable to a body of some other or further medical device, such as an infusion device, an infusion pump, an injection pump, an inhaler, or a portable medical analysis device, such as a blood glucose meter. Insofar, any reference to an injection device may be regarded as a reference to a respective medical device, respectively.

According to another aspect the disclosure also relates to an injection device for setting and injecting of a dose of a medicament. The injection device may be configured as a handheld pen-type injector or as a medicament pump. The injection device includes a housing configured to accommodate a medicament container. The injection device further includes a drive mechanism configured to withdraw or to expel a dose of the medicament from the medicament container. The drive mechanism is further configured to inject the dose of the medicament into biological tissue. The injection device is further provided with a label as described above. The label is attached to the housing or to a body of the injection device. Typically, the body of the injection device is part of the housing of the injection device. It may be a proximal part of the housing.

The drive mechanism of the injection device typically includes a piston rod displaceable along a longitudinal direction. The piston rod is configured to operably engage with a piston of a cartridge containing the injectable medicament. A distal end of the cartridge located opposite to the piston rod is provided with a pierceable seal that is typically penetrable by a double tipped injection needle.

With the label attached to the outside of the housing of the injection device an all mechanically implemented injection device, such as a disposable or reusable injection pen can be retrofitted with a memory aid or reminder thus assisting a user with regards to a proper handling and operation of the injection device.

With further examples, the disclosure relates to a medical device, such as an infusion device, an infusion pump, an injection pump, an inhaler, or a portable medical analysis device, such as a blood glucose meter. The medical device is further provided with a label as described above. The label is attached to the housing or to a body of the medical device. The body of the medical device may be part of the housing of the respective medical device.

According to a further example the medicament container, e.g. in form of a cartridge containing the medicament is arranged inside the housing of the injection device. The injection device may be configured as a disposable injection device. The medicament container filled with the medicament may be readily assembled inside the injection device as the injection device is handed out to customers or patients.

The injection device may be implemented as a disposable injection device intended and/or configured to be discarded in its entirety after use and/or when the medicament has been used up or has exceeded its best before date. With other examples the injection device is implemented as a reusable injection device, wherein a medicament cartridge or medicament container filled with the medicament can be exchanged and replaced.

With some examples the injection device is a fixed dose injection device providing setting and injecting only one of a predefined those size. With other examples the injection device is implemented as a variable dose injection device, wherein a user may individually set doses of different sizes for injection.

In a further aspect the disclosure also relates to an adapter configured for a releasable attachment to a housing of an injection device. The adapter includes a rigid body including an outside surface and including a counter fastening feature configured to releasably mechanically engage with a correspondingly or complementary shaped fastening feature of the housing of the injection device. The adapter further includes a label as described above that is attached to the outside surface of the rigid body. Here, the injection device itself may be void of a label as described above. The injection device may include only a conventionally printed label.

With some examples the rigid body includes a tubular-shaped sleeve configured to receive at least a portion of the housing of the injection device inside the sleeve. The label may be adhesively attached to the outside surface of the rigid body. The label can be prefixed to the outside surface of the rigid body. It is fixed and attached to the rigid adapter body in a well-defined position and/or orientation. The adapter provided with the label on its outside surface is particularly configured to retrofit existing injection devices with a label without the necessity to e.g. adhesively attach the label to the injection device.

With numerous examples and when the label is equipped with a device sensor it may be important that the label is correctly and/or precisely attached and fixed in a well-defined position and/orientation relative to the housing of the injection device. This can be provided with the counter fastening feature of the rigid body configured to engage with the fastening feature of the injection device.

The fastening feature may include at least one of a radial protrusion or radial recess to engage with a correspondingly shaped radial recess or radial protrusion of the counter fastening feature. The fastening feature and the counter fastening feature may be implemented as mutually corresponding snap features. Moreover, only one of the counter fastening feature and the fastening feature may be implemented as a snap feature whereas the other one of the counter fastening feature and the fastening feature is implemented as a raised ridge or groove on the outside of the housing of the injection device or on the inside of the rigid body of the adapter.

With a further example the rigid body of the adapter includes a cap fastening feature configured for engagement with a protective cap of the injection device. This is of particular benefit when the fastening feature of the housing of the injection device is implemented as a cap fastening feature configured to engage with a counter fastening feature of the protective cap of the injection device. The cap fastening feature of the housing of the injection device may be originally configured for a releasable attachment and/or releasable fixing of a protective cap of the injection device, e.g. covering a distal end of the housing of the injection device.

The counter fastening feature of the rigid body may mimic or replace the counter fastening feature of the protective cap of the injection device. The counter fastening feature of the rigid body may engage with the fastening feature of the housing of the injection device instead of the fastening feature of the protective cap. The rigid body, e.g. implemented as a tubular shaped sleeve may be adapted to receive a proximal housing portion of the injection device in such a way, that the counter fastening feature of the rigid body is located at a distal end of the rigid body. Then, the distal end of the rigid body may be provided with a cap fastening feature to engage with a counter fastening feature of the protective cap. Here, the protective cap originally supplied with the injection device may be replaced by another protective cap configured for engagement with the rigid body of the adapter when the adapter is engaged with the Fastening feature of the housing of the injection device.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound includes at least one peptide for the treatment and/or prophylaxis of diabetes mellitus or complications associated with diabetes mellitus such as diabetic retinopathy,

wherein in a further embodiment the pharmaceutically active compound includes at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure. The label, the injection device and the adapter are not limited to specific embodiments or examples but include any combination of elements of different embodiments or examples. Insofar, the present disclosure covers any combination of claims and any technically feasible combination of the features disclosed in connection with different examples or embodiments.

### BRIEF DESCRPTION OF THE FIGURES

In the following, numerous examples of a container and of an injection device will be described in greater detail by making reference to the drawings, in which:

FIG. 1 shows an example of an injection device,

FIG. 2 shows the injection device of FIG. 1 in an exploded perspective view,

FIG. 3 shows the injection device of FIG. 1 with a touch sensitive label attached to the housing of the injection device,

FIG. 4 is an enlarged view of one configuration of the label,

FIG. 5 is a further illustration of the label in another configuration,

FIG. 6 shows a further configuration of the label,

FIG. 7 shows another configuration of the label,

FIG. 8 shows a further configuration of the label with the first and second information content in a flipped or turned orientation,

FIG. 9 is a perspective illustration of an injection device provided with an adapter carrying the label, wherein the label is in a pre-assembly configuration,

FIG. 10 is illustrative of a longitudinal cross-section through the assembly of the injection device with the adapter,

FIG. 11 shows the multilayer structure of the touch sensitive label,

FIG. 12 is a cross-section through an example of the touch sensitive label,

FIG. 13 is a block diagram illustrating the electronic circuit of the touch sensitive label,

FIG. 14 shows another example of the touch sensitive label equipped with a sensor, and

FIG. 15 shows a cross-section through the injection device with the touch sensitive label attached thereto.

### DETAILED DESCRIPTION

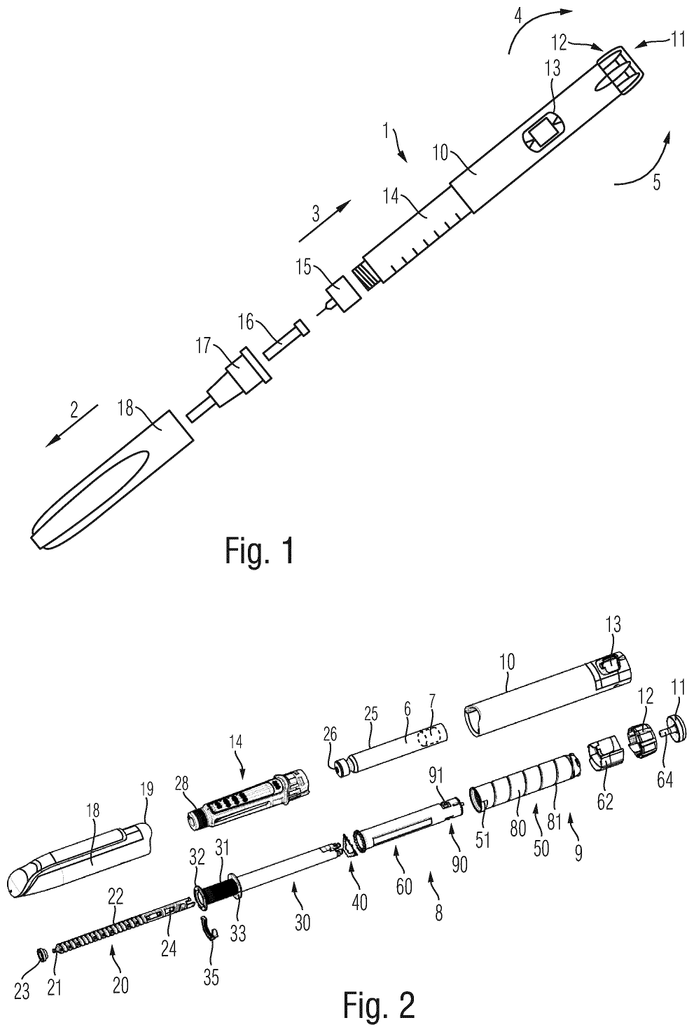

The injection device 1 as shown in FIGS. 1 and 2 is a pre-filled disposable injection device that includes a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 may include and form a main housing part configured to accommodate a drive mechanism 8 as shown in FIG. 2. The injection device 1 may further include a distal housing component denoted as cartridge holder 14. The cartridge holder 14 may be permanently or releasably connected to the main housing 10. The cartridge holder 14 is typically configured to accommodate a cartridge 6 that is filled with a liquid medicament. The cartridge 6 includes a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder 14 includes a threaded socket 28 at its distal end to threadedly engage with a correspondingly threaded portion of the injection needle 15. By attaching the injection needle 15 to the distal end of the cartridge holder 14 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 μg of pure crystalline insulin (1/22 mg). The dose dial 12 may include or may form a dose dial.

As shown further in FIGS. 1 and 2, the housing 10 includes a dosage window 13 that may be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The number sleeve 80 mechanically interacts with a piston in the insulin cartridge 6. When the needle 15 is stuck into a skin portion of a patient, and when the trigger 11 or injection button is pushed, the insulin dose displayed in display window 13 will be ejected from injection device 1. When the needle 15 of the injection device 1 remains for a certain time in the skin portion after the trigger 11 is pushed, a high percentage of the dose is actually injected into the patient's body. Ejection of an insulin dose may also cause a mechanical click sound, which is however different from the sounds produced when using the dose dial 12.

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

Furthermore, before using injection device 1 for the first time, it may be necessary to perform a so-called “prime shot” to remove air from the cartridge 6 and the needle 15, for instance by selecting two units of the medicament and pressing trigger 11 while holding the injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

An example of the drive mechanism 8 is illustrated in more detail in FIG. 2. It includes numerous mechanically interacting components. A flange like support of the housing 10 includes a threaded axial through opening threadedly engaged with a first thread or distal thread 22 of the piston rod 20. The distal end of the piston rod 20 includes a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 includes an inner thread threadedly engaged with the proximal thread 24 of the piston rod 20. Moreover, the drive sleeve 30 includes an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further includes a radial recess or protrusion at its outer circumference to engage with a complementary-shaped recess or protrusion at an inside of the sidewall of the housing 10. In this way the last dose limiter 35 is splined to the housing 10. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number 80 to prevent relative movement there between. The dose dial 12 is provided with a central opening.

The trigger 11, also denoted as dose button is substantially T-shaped. It is provided at a proximal end of the injection device 10. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40 also acting as a clicker and the clutch 60 engaged, the drive sleeve 30, the spring or clicker 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first sense of rotation, e.g. in a dose incrementing direction 4. Rotation of the piston rod 20 is prevented due to the opposing directions of the overall and driven threads on the piston rod 20.

The last dose limiter 35 keyed to the housing 10 or body is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch may serve as a ratchet mechanism.

As an alternative or in addition the ratchet mechanism 90 may include at least one ratchet feature 91, such as a flexible arm on the sidewall of the tubular-shaped clutch 60. The at least one ratchet feature 91 may include a radially outwardly extending protrusion e.g. on a free end of the flexible arm. The protrusion is configured to engage with a correspondingly shaped counter ratchet structure on an inside of the number sleeve 80. The inside of the number sleeve 80 may include longitudinally shaped grooves or protrusions featuring a saw-tooth profile. During dialing or setting of a dose the ratchet mechanism 90 allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism 90 provide a torque transmission from the number sleeve 80 to the clutch 60.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

In the presently illustrated example, the number sleeve 80 represents a dose tracker 50 being indicative of a size of a dose currently set. Here, the longitudinal and/or rotational position of the dose tracker 50 relative to the housing 10 or body of the injection device 1 is indicative of a size of a currently set. The dose tracker 50 includes a tracking stop feature 51 that is operable to engage with a counter stop of the housing 10, e.g. when a zero-dose configuration or when a maximum dose configuration has been reached.

The number sleeve 80 is only one example of a dose tracker 50. Likewise, also the piston rod 20 may act as or may be used as a dose tracker 50.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

The dose setting mechanism 9 as illustrated in FIG. 2 includes at least the dose dial 12 and the number sleeve 80. As the dose dial 12 is rotated during and for setting of a dose the number sleeve 80 starts to rotate relative to the housing along a helical path as defined by the threaded engagement of its outer thread or helical groove 81 with a correspondingly shaped threaded section at the inside surface of the housing.

During dose setting and when the drive mechanism 8 or the dose setting mechanism 9 is in the dose setting mode the drive sleeve 30 rotates in unison with the dose dial 12 and with the number sleeve 80. The drive sleeve 30 is threadedly engaged with the piston rod 20, which during dose setting is stationary with regard to the housing 10. Accordingly, the drive sleeve 30 is subject to a screwing or helical motion during dose setting. The drive sleeve 30 starts to travel in proximal direction as the dose dial is rotated in a first sense or rotation or in a dose incrementing direction 4, e.g. in a clockwise direction. For adjusting of or correcting a size of a dose the dose dial 12 is rotatable in an opposite second sense of rotation, hence in a dose decrementing direction 5, e.g. counterclockwise.

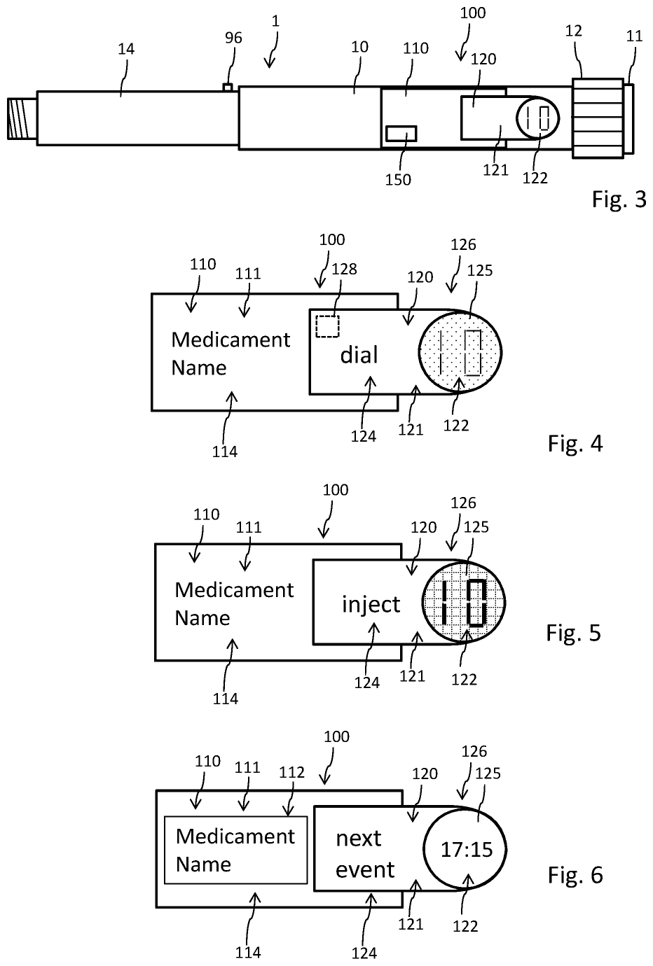

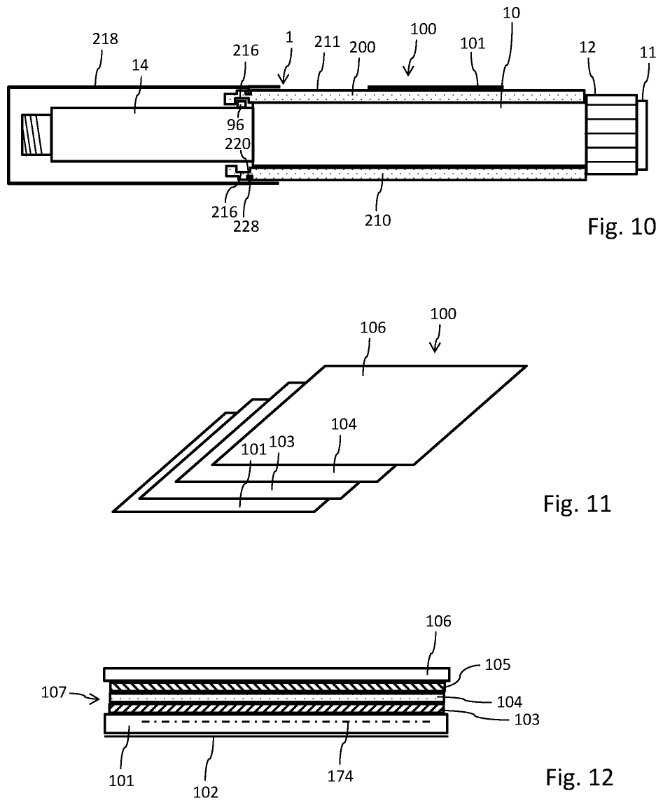

In FIGS. 3-15 numerous examples of a label 100 configured for attachment to a tubular shaped housing 10 or body of the injection device 1 are illustrated. As further indicated in FIGS. 9 and 10 the label 100 for the injection device 1 may be configured for attachment to an outside surface 211 of a rigid body 210 of an adapter 200, wherein the adapter 200 is configured to releasably and mechanically engage with a fastening feature 96 of the housing 10 of the injection device 1.

For this the adapter 200 includes a counter fastening feature 216 correspondingly or complementary shaped to the fastening feature 96.

The label 100 includes a flexible substrate 101 as illustrated in FIG. 11. On the flexible substrate 101 there are provided a first label area 110 and a second label area 120 as illustrated in greater detail in the sequence of FIGS. 3-8. The first label area 110 is provided with a first information content 111. The first information content 111 includes at least one visual sign 114 or character. The second label area 120 is non-overlapping with the first label area 110.

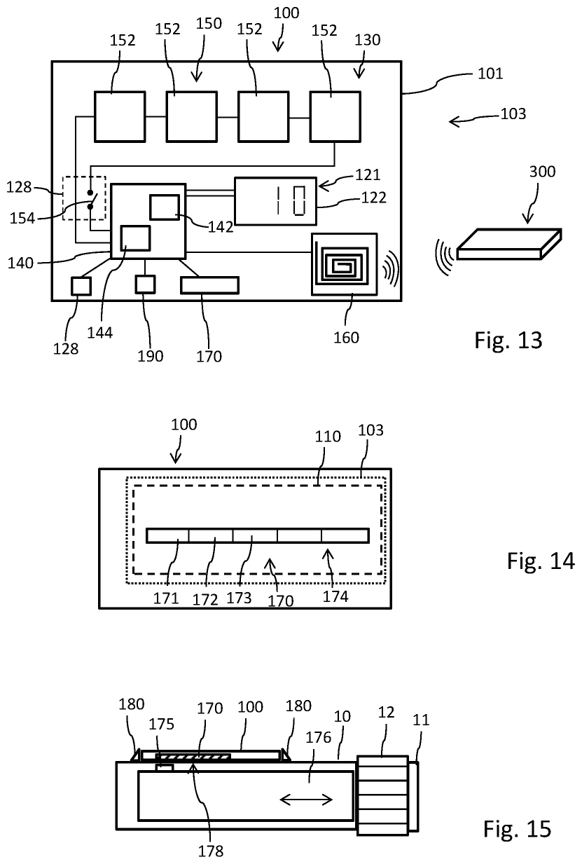

The second label area 120 is configured to display or to provide a second information content 121. The first information content 111 and the second information content 121 are different. The second label area 120 includes an electronic display 122. The electronic display 122 is configured to display the second information content 121. The label 100 further includes a processor 140 as schematically illustrated in the block diagram of the label 100 of FIG. 13. The processor 140 is connected to the electronic display 122. The processor 140 is configured and operable to modify at least the second information content 121 illustrated on or displayed by the electronic display 122.

Typically, the first and second label areas 110, 120 are located on an upper side of the substrate 101. The label 100, at least the electronic display 122 of the label 100 includes a multilayer structure as indicated in FIGS. 11 and 12. The electronic display 122 may thus include a multilayer structure, e.g. a thin film multilayer structure.

The flexible substrate 101 or base substrate may be any material known for producing printed electronic labels, such as PET film or office paper. Other plastic materials are also feasible, e.g. PVC. The base substrate has an adhesive on one side to fix the label to a pen body, e.g. either permanently or removeably, depending on the choice of adhesive.

The label 100 includes a mechanically flexible structure. The label 100 is bendable or wrappable around an outer circumference or outside surface of the body 10 of the injection device and/or of a rigid body 210 of the adapter 200, the latter one of which being illustrated in FIGS. 9 and 10.

Hence, the electronic display 122 is a flexible electronic display. It may include one of an electroluminescent display, an electrophoretic display, a liquid crystal display and a light emitting diode display, in particular an organic light emitting diode display (OLED). Such displays are known to be flexible as well as bendable.

Optionally, the label 100, e.g. the second label area 120 may be provided with an input 128, e.g. implemented as a touch sensitive area of the second label area 120. The touch sensitive area or the input 128 may be implemented as a portion of the second label area 120. With some examples the entirety of the electronic display 122 may be implemented as a touch sensitive area 128. Hence, the touch sensitive area 128 may overlap with the electronic display 122. In other words, the electronic display 122 may be implemented as a touch sensitive electronic display.

The input 128 is connected to the processor 140. By mechanically engaging, e.g. by touching the input 128 the processor 140 can be prompted to modify or to alter the second information content 121 on the second label area 120. With some examples the input 128 or the touch sensitive area of the electronic display 122 includes capacitive switches produced in printing electronics technology. The input 128 can be implemented as an electrode, which is part of a capacitor. The capacitor can be detuned when an object, such as a finger approaches and comes in close vicinity to the capacitor. This approach has an influence on a measurable property of the capacitor, e.g. on the capacitance thereof. This modification of the capacitor is measurable as/or detectable by the processor 140.

With some embodiments as for instance illustrated in FIGS. 4 and 5 the first information content 111 is printed or imprinted on the first label area 110. Here, the first information content 111 may include a printing color or an ink directly or indirectly provided on an upper or outside surface of the flexible substrate 101. There may be provided a printed layer on the flexible substrate 101 that is particularly suitable for adhesion of a printing ink or the like printable and visual substance used for printing or implementing the first information content 111 on the first label area. As illustrated in FIGS. 4-8, the first information content 111 includes at least one visual sign 114 or character. With the presently illustrated examples the first information content 111 includes information about the medicament or medicament type, such as the medicament name in the first label area 110.

Typically and with the examples as illustrated in FIGS. 4-8 the first information content 111 is typically indicative or contains information about the medicament or about generic properties of the medicament. The first information content 111 may include information such as the medicament type, the medicament name, the medicament concentration, a manufacturing date, a best before date, a manufacturer name, a manufacturing site and/or a LOT number.

When printed or imprinted on the first label area 110 the first information content 111 is persistent and durable. It is non-erasably or non-modifiably provided on the first label area 110. In this way, the label 100 may fulfill regulatory requirements in terms of labeling of medicaments.

The second information content 121 illustrated and provided in the second label area 120 is modifiable by the processor 140. The electronic display 122 is typically configured to visualize or to illustrate at least one visual sign 124. The visual sign 124 may include an instruction to a user, such as dial, set, inject, hold, injection completed, due date or due time. In addition or alternative the electronic display 122 may visually illustrate a size of a dose, e.g. a sequence of digits or numbers being indicative of e.g. international units of a size of a dose of the medicament to be set, to be dialed and/or to be injected. In addition, the second information content 121 may include a date or a point of time indicating to a user at which time the next event or the next use of the injection device is currently due. Moreover, the second information content 121 may include a time interval or a duration, e.g. indicating a holding time or a remaining holding time during which an injection needle should remain in the injection site of a patient after a dose injection process has completed.

As it becomes apparent from a comparison of FIGS. 4 and 5 the electronic display 122 is a color display 126. The color of at least one of the information background 125 and the visual sign 124 presented on the electronic display 122 can be modified. In FIGS. 4 and 5 the different patterns of the information background 125 represent different colors, different brightness or different contrast. Moreover, the color, the brightness and the contrast of visual signs 124 as displayed or provided on the electronic display 122 may be subject to respective modifications on demand.

In the configuration of FIG. 4 the second information content 121 indicates to a user, that a dialing or setting of a dose is currently due. The label 100 may include a device sensor 170 the details of which being described below in order to determine or to measure a size of a dose currently set with the injection device 1 when the label 100 is attached to the housing 10 of the injection device 1. When the label 100 has detected or measured that a prescribed dose has been dialed, the second information content 121 may reflect or may indicate the correct dose dialing. It may provide a respective confirmation, e.g. by changing the color of at least one of the visual sign 124 and the information background 125. E.g., the color may switch from red to green. Alternatively or additionally, at least one of the brightness and contrast of the information content 121 may be subject to a respective modification.

Moreover or alternatively, the information content 121 may blink in order to visually indicate to a user, that a prescribed size of a dose has been correctly set. In FIG. 5 a situation is illustrated, in which after setting of a correct dose the label informs the user to trigger a dose injection, e.g. by depressing the trigger 11.

After termination of the injection procedure the information content 121 may switch or alter again. It may indicate to a user, that the injection needle should remain inside the pierced skin by a holding time. The holding time of e.g. 5 seconds or 10 seconds may be visually illustrated in the information content 121. The illustrated holding time may be dynamically illustrated to a user of the label 100. The holding time may include a countdown scheme and may visualize the currently remaining holding time until the injection needle can be withdrawn from the injection site of the patient.

Thereafter the information content 121 may be modified again and may indicate to a user, that the injection has been completed. Thereafter the information content 121 may switch into a configuration as illustrated in FIG. 6. There, the information content 121 is illustrative of a point of time at which the next event, e.g. the next injection of the medicament is due.

In FIG. 6 a further implementation of the flexible label 100 is illustrated. There, the first label area 110 includes a first electronic display 112. The electronic display 122 then has to be regarded and denoted as a second electronic display. The first electric display 112 may be provided with an information content 111. As before, the information content 111 includes at least one visual sign or character 114.

Typically, the first electronic display 112 and the second electronic display 122 are of different display type. The first electronic display 112 is of a first display type and the second electronic display 122 is of a second display type. The first display type and the second display type are different display types. The first display 112 is one of an electroluminescent display, an electrophoretic display, a liquid crystal display and a light emitting diode display, in particular an organic light emitting diode display. Also the second display 122 is one of an electroluminescent display, an electrophoretic display, a liquid crystal display and a light emitting diode display, in particular an organic light emitting diode display. With some examples the first display 112 and hence the first display type is an electrophoretic display and the second display 122 or the second display type is a light emitting diode display, in particular an organic light emitting diode display.

The electrophoretic display of the first display 112 is particularly configured to consistently provide medicament related information in order to conform with regulatory requirements in terms of medicament labeling. For this, the electrophoretic display is of particular advantage. Even if the label should be void of electric energy the first information content 111 is still and persistently visible in the first label area 110. The electrophoretic display of the first electronic display 112 provides excellent readability with high contrast even when subject to a rather direct illumination of a bright light source, such as sunlight.

When the first label area 110 is provided with a first electronic display 112 the electronic display 112 may be also connected to the processor 140. In this way also the first information content 111 of the first label area may be modified by the processor 140. Modification of first and/or second information contents 111, 121 may be triggered quasi-automatically, e.g. after lapse of a predefined time interval. The modification of the first and/or second information contents 111, 121 may be triggered by a user input, e.g. by activating the input 128.

Moreover, a modification of the information content 111, 121 may be triggered by at least one of a device sensor 170 and an ambient sensor 190 connected to the processor 140. Here, at least one of the first and the second information contents 111, 121 is modifiable in response to signals received from at least one of the device sensor 170 and the ambient sensor 190. In this way, the label 100 dynamically reacts to a variety of ambient conditions or to varying configurations of the injection device 1.

The ambient sensor 190 integrated into the label 100 and/or arranged on the flexible substrate 101 is one of an ambient brightness sensor, and acceleration sensor, an orientation sensor, a temperature sensor or an air pressure sensor. When implemented as an orientation sensor, acceleration sensor or ambient air pressure sensor, the ambient sensor 190 is configured to generate at least one electrical signal when the label is subject to an acceleration, a movement or reorientation with regard to the field of gravity.

In response to the generation of such sensor signals the processor 140 may be configured wake up and may activate or switch on the second electronic display 122. In this way, a quasi-automated wake-up or quasi-automated activation of the label 100 can be implemented.

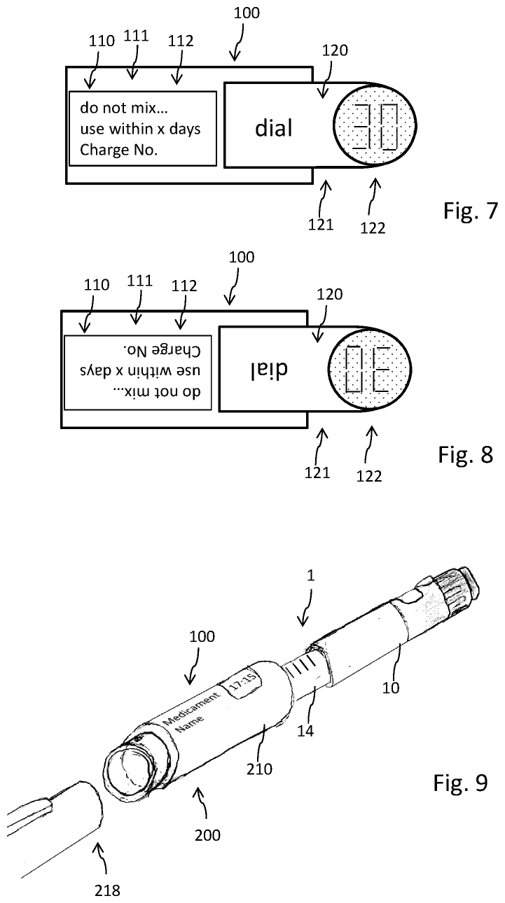

In response to sensor signals obtained from the ambient sensor 190 at least one of the first electronic display 112 and the second electronic display 122 may change its appearance. In an idle or sleep mode the label 100 may provide basic information about the medicament in the first label area 110. Here, the first information content 111 may provide basic medicament-related information in the first label area 110, which medicament information being conform with regulatory provisions. Upon wake up and/or activation of the label through signals generated and/or provided by the ambient sensor 190 the processor 140 may be configured to alter or to change the first information content 111 and/or the second information content 121. As it is for instance apparent by a comparison of the first information content 111 as illustrated in FIGS. 6 and 7 the medicament name may disappear on behalf of other medicament-related information, such as use instructions or information regarding use, pharmaceutical stability or shelf life of the medicament.

Upon wake up or activation of the label 100 also the second information content 121 may be subject to a respective modification.

Moreover and as illustrated by a comparison of FIGS. 7 and 8 the processor 140 is operable to turn or to flip an orientation of at least one of the first information content 111 and the second information content 121 on the basis of an electric signal received from the ambient sensor 190. Here, the ambient sensor 190 may be implemented as an acceleration sensor or orientation sensor. The momentary orientation of the first information content 111 and/or of the second information content 121 with regard to the local field of gravity can be determined. If the label 100 should be oriented upside down the processor 140 is configured to flip or to turn the orientation of the first information content 111 and/or of the second information content 121 accordingly as illustrated in FIG. 8.

In this way, and when the label 100 is attached in a predefined way to the injection device 1 the label 100 and the injection device 1 are likewise usable by right-handed and left-handed persons. When used by left-handed persons the information content 111, 121 of the label 100 is turned upside down compared to configurations of the label 100 when the respective injection device 1 is used by right-handed persons or patients.

The example of an electronic circuit 130 of FIG. 13 is by no way limiting for the examples of FIGS. 3-15. The electronic display 112 and/or the electronic display 122 may superimpose the electronic circuit 130. Hence, at least one or both of the electronic displays 112, 122 may entirely overlap with the electronic circuit 130 located underneath. At least one of both of the electronic displays 112, 122 may include a multilayer structure as indicated in FIGS. 11 and 12. The electronic circuit 130 may be implemented in one or several of the various layers 103, 104, 105, 106 of the flexible label 100. Typically, the electronic circuit 130 is entirely provided by a conductive layer 103.

The input 128 or touch sensitive area may be visually indicated on the electronic display 112, 122 or on a separate portion outside the electronic displays 112, 122. The input 128 and the electronic display 112, 122 are configured to interact in such a way, that upon touching or depressing of the input 128, i.e. the touch sensitive area at least the second electronic display 122 changes its visual appearance. The electronic displays 112, 122 may be switchable between a default mode or idle mode and an activated mode.

In a further example and upon touching depressing of input 128 the processor 140 may be configured to record a user activity and/or a point of time of a user activity in the electronic storage 144 or memory of the electronic circuit. The electronic storage 144 may include capacity to store data relating to a plurality of events including time stamps, e.g. 30, 100, 1000 events.

In a further example the electronic circuit includes an antenna providing a data transmission element, e.g. enabling NFC, WIFI or RF data transmission. This way data stored in the memory can be read out by an external device using a respective wireless communication protocol. NFC or RF communication could be implemented in passive or active way, wherein the latter requires an energy source, e.g. battery, powering the electronics. The battery may be implemented in printing technology.

FIG. 13 shows one example of an electronic circuit 130 of the touch sensitive label 100. The electronic circuit 130 may be directly printed on the flexible substrate 101. The electronic structures and/or the conductive structures of the electronic circuit 130 might be bendable or flexible as well. The integrity or functionality of the electronic circuit 130 is substantially unaffected by a bending or flexing of the flexible substrate 101 and/or of the electronic circuit 130.

The electronic circuit 130 includes a battery 150 typically equipped with numerous battery cells 152. The individual battery cells 152 are electrically connected. They may be connected in series or parallel depending on the voltage provided by the individual battery cells 152 and depending on the voltage required by the processor 140. The battery 150 and/or its battery cells 152 may include a printed electronic structure. Hence, the battery 150 and/or the battery cells 152 our printed batteries or battery cells and may be arranged on the substrate 101 by way of printing.

The processor 140 is connected to the battery 150 as well as to the electronic display 122. The interconnection between the battery 150 and the processor 140 may be interrupted by the switch 154 coinciding with the input 128. Depressing of the switch 154 may either connect or disconnect the electrical connection between the battery 150 and the processor 140.

The processor 140 includes a central processing unit (CPU) 142 and a storage 144. In the storage 144 numerous predefined information contents 111, 121 for illustration with at least one of the electronic display 112, 122 may be stored. Upon registration of a closing or opening of the switch 154 the respective information content 121 might be illustrated or displayed on the electronic display 122. When equipped with a data storage 144 the processor 140 may be further configured to count a number of touch operations of the touch sensitive area. If the processor 140 is further equipped with a clock every input or touch instant can be further assigned with a timestamp thus allowing to record a dosing history or to record the points in time at which the input 128 was appropriately touched by the user of the injection device 1.

The electronic circuit 130 may further include an antenna 160 connected to the processor 140. The antenna 160 may be configured for wireless data transmission. The antenna 160 may be configured as a receiving antenna and/or as a broadcasting antenna. The antenna 160 may be configured to transmit electromagnetic signals in the RF frequency band. The antenna 160 may include an RFID antenna. The antenna 160 may be configured in accordance to conventional wireless communication standards, such as Bluetooth, NFC or IEEE 802.11 (WLAN). The antenna 160 is configured to exchange data with an external electronic device 300, such as a smart watch, a smartphone, a tablet computer or a personal computer.

The processor 140 may be reconfigurable by signals obtained from the external electronic device 300 via the antenna 160. In this way the external electronic device 300 can be used to modify or to reconfigure the processor 140 and hence to modify and to reconfigure the content of at least one of the first and second electronic displays 112, 122. In addition or alternative the external electronic device 300 may be further configured to read out the data storage 144 of the electronic circuit 130. In this way the dosing history and the use of the label 100 can be precisely monitored and transmitted to the external electronic device 300 for further processing and/or evaluation.

The entirety of the electronic components of the electronic circuit 130, e.g. the wired connections between the battery 150 and the battery cells 152, the switch 154 or the touch sensitive area forming the input 128, the antenna 160 as well as the processor 140 may include or may be formed by a printing process on the substrate 101. In this way a separate assembly and arrangement of numerous electronic components on the substrate 101 becomes substantially superfluous. This enables a costefficient mass manufacturing of the touch sensitive label 100.

A lower side of the substrate 101 may be provided with an adhesive. The adhesive may be provided on an adhesive layer 102 entirely or at least partially covering the lower side of the substrate 101 located opposite to the conductive layer 103 on which the electronic circuit 130 is located. In FIG. 12 a stack of numerous layers 103, 104, 105, 106 configured to form one of the electronic displays 112, 122 is exemplary illustrated. The stack structure of FIG. 12 represents a thin film electroluminescent display 112. The substrate 101 may be pliable and may include or consist of one of the following: foldable office paper, transparent or non-transparent PET film, leather, wood, ceramics, and a metal foil. The electroluminescent display is configured to actively emit light.

A segment of the display consists of two overlaid electrodes that act as a capacitor. The oppositely located electrodes are provided in the conductive layer 103 and in the transparent electrode layer 106. Between these layers 103, 106 there is provided a dielectric layer 104 and an electroluminescent layer 105, e.g. in form of a phosphor layer. If a suitable voltage and a suitable current AC signal is applied the electroluminescent layer 105 emits photons.

The stack of layers 103, 104, 105, 106 may add only 100-150 pm of thickness to the substrate 101. In this way the electronic display 110 can be extraordinarily thin.

With other examples the flexible electronic display 110 is implemented as an electrophoretic display that is based on rearranging charged pigment particles by means of an applied electric field. There, titanium dioxide particles of appropriately 1 μm in diameter may be dispersed in a hydrocarbon oil. A dark colored dye is added to the oil along with surfactants and charging agents that cause the particles to take on an electric charge. This mixture is placed between two parallel, conductive plates separated by a gap of 10-100 μm. When a voltage is applied across the two plates the particles migrate electrophoretically to the plate that bears the opposite charge from that on the particles.

When the particles are located at the front or a viewing side of the display, it appears white because light is scattered back to the viewer by the high index titania particles. When the particles are located at the rear side of the display it appears dark because the incident light is absorbed by the colored dye. If the rear electrode is divided into a number of small picture elements or pixels, an image can be formed by applying the appropriate voltage to each region of the display to create a pattern of reflecting and absorbing regions. Electrophoretic displays are considered prime examples of an electronic paper category because of their paperlike appearance and lower power consumption.

The label 100 may only be optionally equipped with an antenna 160. With one implementation the label 100 may be void of an antenna 160 and may be operable to illustrate a well-defined or predefined information content 111 and to provide a switching between a sleep mode or activated mode.

In FIGS. 14 and 15 there is illustrated a further example of a flexible label 100. This label 100 may be implemented with or without an antenna 160. It includes a flexible substrate 101 with at least one electronic display 112, 122. Typically, the label 100 of FIG. 14 also includes a processor 140, a CPU 142, a data storage 144 and a battery 150 as described above in connection with FIG. 13. In addition, the label 100 of FIGS. 14 and 15 includes a device sensor 170.

The device sensor 170 is configured to allocate or to determine a position and/or a rotational state of a dose tracker 176 of the injection device 1 when the label 100 is attached to the housing 10 in a predefined manner. Here, the dose tracker 176 may coincide or may represent the dose tracker 50 as described above in connection with FIGS. 1-2. Alternatively, the dose tracker 176 may be represented by any other component of the injection device 1. The dose tracker 176 may be represented e.g. by the piston rod 20 or by some arbitrary component of the drive mechanism 8 or dose setting mechanism 9, wherein the position and/or orientation of the respective device component relative to the housing 10 is indicative of a size of a dose currently set or dispensed.

For arranging the label 100 to the housing 10 the housing 10 may include at least one or several position marks 180 illustrated in FIG. 15 as protrusions on the outside surface of the housing 10. The label 100 has to be properly attached to the housing 10 in the area as defined by the at least one or several position marks 180. The dose tracker 176 may coincide with the number sleeve 80 of the injection device 1 or may be formed by the number sleeve. The dose tracker 176 includes an indicator 175 whose positional state, i.e. the longitudinal position and/or a rotational orientation relative to the housing 10 is detectable by the device sensor 170.

The position mark 180 may protrude from the sidewall of the housing 10 or may include a recess in the housing 10. Alternative, the position mark 180 may be void of protrusions or recesses in the outside surface of the housing 10. The position mark 180 may simply include a visual indication, such as a border region inside which the label 100 should be fastened, e.g. adhesively attached.

In one example the device sensor 170 includes numerous discrete sensor segments 171, 172, 173 that are separated along a moving direction of the indicator 175 and/or of the dose tracker 176 relative to the housing 10. As the dose tracker 176 is subject to a rotational and/or sliding movement relative to the housing 10 the indicator 175, e.g. initially overlapping with a first sensor segment 171 moves towards a second sensor segment 172 and, e.g. further towards the third sensor segment 173. The movement of the indicator 175 relative to the numerous sensor segments 171, 172, 173 is detectable by the device sensor 170 that is electrically connected to the processor 140. In this way, the processor 140 and the device sensor 170 are configured to determine and to detect an actual position and/or rotational state of the indicator 175 and hence of the dose tracker 176 relative to the housing 10.

The position or rotational state of the dose tracker 176 unequivocally coincides with a size of a dose actually set by the injection device 1. In this way, the processor 140 may be configured to determine or to measure a size of a dose actually set with the injection device 1 when the label 100 is appropriately connected to the housing 10. The determined longitudinal or rotational position of the dose tracker 176 may be thus compared with a predefined position, e.g. indicated by the second information content 111 on the electronic display 112. The dose actually set with the injection device may be further illustrated through the dosage window 13.

The specific implementation of the device sensor 170 and the indicator 175 may vary. As illustrated in FIG. 15 the housing 10 may include a through opening 178 or a recess through which the position of the indicator 175 can be for instance mechanically or electrically detected, e.g. by means of numerous contact pins provided on each sensor segment 171, 172, 173. For this, there may be established a direct mechanical contact between the indicator 175 and at least one of the sensor segments 171, 172, 173.

With other examples the indicator 175 may be magnetically encoded and the sensor segments 171, 172, 173 may be configured to detect a magnetic field of the indicator 175 as the indicator 175 is subject to a longitudinal and/or rotational movement. With a further example the indicator 175 and the sensor segments 171, 172, 173 may be implemented electrostatically. Here, the numerous sensor segments 171, 172, 173 may be configured to allocate or to detect a modification of an electric field induced by the indicator 175. Furthermore, the sensor segments 171, 172, 173 may include capacity measuring elements configured to measure a modification of an electric field or electric capacitance in the vicinity of the respective sensor segments 171, 172, 173. Magnetic, electrostatic and capacitive measurement procedures may be of particular benefit because they may not require a through opening 178 or recess in the sidewall of the housing 10. With such implementations the label 100 may be simply adhesively attached within the given position marks on the outside surface of the housing 10.

With the example as illustrated in FIGS. 11 and 12 there may be provided at least one electrode structure 174 of a device sensor 170 on the flexible substrate 101. At least one or all of the sensor segments 171, 172, 173 as described above may belong to or may constitute the electrode structure 174. The electrode structure 174 may be located between the adhesive layer 102 and at least one of the conductive layers 103, 106. The electrode structure 174 may belong to the device sensor 170 or it may be part of the device sensor 170. The electrode structure 174 on the substrate 101 is configured to measure at least one of an electric capacitance, an electric field and a magnetic field in the vicinity of the substrate 101. Typically, the electrode structure 174 is configured to determine or to quantitatively measure a position and/or a rotational state of at least one device component, e.g. of a dose tracker 176 of the injection device 1.

With further examples the electrode structure 174 may be configured to determine a longitudinal position of the piston rod 20 and/or the longitudinal position of the bung 7 or piston of the cartridge 6 with regards to a barrel of the cartridge 6 and/or with regards to the housing or body 10 of the injection device 1.

At least one or both of the conductive layer 103 and the transparent electrode layer 106 include(s) an electrically conductive grid electrode at least partially spatially overlapping with the electrode structure 174 of the device sensor 170. Here, and due to the spatial overlap of the conductive grid electrode 103, 106 with the electrode structure 174 of the device sensor 170 the electrically conductive grid electrode 103, 106 provides an electromagnetic shield 107 for the device sensor 170. In this way, the electromagnetic compatibility (EMC) of the label 100 can be increased and the position or orientation of the at least one dose tracker 176 can be measured and defined with high precision.

As illustrated in FIG. 14, the electrically conductive grid electrode 103 spatially overlaps with the electrode structure 174 and the numerous sensor segments 171, 172, 173 of the device sensor 170.

In FIGS. 9 and 10 an adapter 200 configured for a releasable attachment to the housing or body 10 of the injection device 1 is illustrated. The adapter 200 includes a rigid body 210. The rigid body 210 includes a tubular-shaped hollow sleeve sized to receive at least a distal portion of the housing 10 of the injection device 1. In particular, the inner diameter of the body 210 is sized to receive the cartridge holder 14 of the injection device 1. The rigid body 210 and hence the sleeve may be also sized to receive the proximal housing or body 10 of the injection device 1 as illustrated in FIG. 10. In a final assembly configuration as illustrated in FIG. 10, a proximal end of the rigid body 210 may be located adjacent or may adjoin a distal end of the dose dial 12.

The adapter 200 and its body 210 includes at least one counter fastening feature 216 to engage with a fastening feature 96 of the housing 10 of the injection device 1. As illustrated in FIG. 10, the fastening feature 96 may include a radially outwardly extending protrusion. The protrusion may include at least one of a pin and a radially outwardly extending rim, e.g. on a proximal end of the cartridge holder 14. Typically, the fastening feature 96 is configured to engage with a respective counter fastening feature of the protective cap 18 as illustrated in FIGS. 1 and 2.

Now and for fastening the adapter 200 to the housing 10 of the injection device the adapter 210 includes the counter fastening feature 216 on an inside of its hollow sleeve. The counter fastening feature 216 is typically arranged at or near the distal end of the rigid body 210. The counter fastening feature 216 may include a recess or a groove on the inside surface of the elongated sleeve of the rigid body 210. In this way the counter fastening feature 216 and the fastening feature 96 may engage by way of a snap fit engagement. They may engage frictionally or by way of a form fit. In this way, the body 210 of the adapter 200 can be fastened in a well-defined and precise manner on the outside of the housing 10 of the injection device 1. The body 210 includes an outside surface 211 on which the label 100 as described above is rigidly or detachably fastened. Typically, the label 100 is adhesively attached to the outside surface 211.

Since the adapter 200 is engageable with the fastening feature 96 of the injection device 1, which is originally intended for fastening of the protective cap 18 as illustrated in FIGS. 1 and 2, the protective cap 18 is replaceable and/or is actually replaced or substituted by a replacement protective cap 218. The replacement protective cap 218 is slightly larger in size at least in the region where it overlaps with the fastening feature 96 of the housing or body 10. The body 210 of the adapter 200 includes a cap fastener 220 configured and operable to engage with a counter cap fastener 228 provided on the replacement protective cap 218.

At least one of the cap fastener 220 and the counter cap fastener 228 includes a radial protrusion complementary shaped to a respective radial recess of the other one of the cap fastener 220 and the counter cap fastener 228. In this way, the replacement protective cap 218 can be mounted and fastened to a distal portion or distal end of the adapter 200 when the adapter 200 occupies the original fastening feature 96 of the housing 10 of the injection device 1.

### REFERENCE NUMBERS

1 injection device

2 distal direction

3 proximal direction

4 dose incrementing direction

5 dose decrementing direction

6 cartridge

7 bung

8 drive mechanism

9 dose setting mechanism

10 housing

11 trigger

12 dose dial

13 dosage window

14 cartridge holder

15 injection needle

16 inner needle cap

17 outer needle cap

18 protective cap

19 protrusion

20 piston rod

21 bearing

22 first thread

23 pressure foot

24 second thread

25 barrel

26 seal

28 threaded socket

30 drive sleeve

31 threaded section

32 flange

33 flange

35 last dose limiter

36 shoulder

40 spring

41 recess

50 dose tracker

51 tracking stop feature

60 clutch

62 insert piece

64 stem

80 number sleeve

81 groove

90 ratchet mechanism

91 ratchet feature

95 preselector

96 fastening feature

100 label

101 substrate

102 adhesive layer

103 conductive layer

104 dielectric layer

105 electroluminescent layer

106 transparent electrode layer

107 electromagnetic shield

108 recess

110 label area

111 information content

112 electronic display

114 visual sign

120 label area

121 information content

122 electronic display

124 visual sign

125 information background

126 color display

128 input

130 electronic circuit

140 processor

142 CPU

144 storage

150 battery

152 battery cell

154 switch

160 antenna

170 device sensor

171 sensor segment

172 sensor segment

173 sensor segment

174 electrode structure

175 indicator

176 dose tracker

178 through opening

180 position mark

190 ambient sensor

200 adapter

210 body

211 outside surface

216 counter fastening feature

218 protective cap

220 cap fastener

228 counter cap fastener

300 electronic device

1-16. (canceled)

17. A label for an injection device, the label comprising:
a flexible substrate configured for attachment to a body,
a first label area on the substrate provided with a first information content, the first information content comprising at least one visual sign or character,
a second label area on the substrate non-overlapping with the first label area , wherein the second label area comprises an electronic display configured to display a second information content, and
a processor connected to the electronic display and configured to modify at least the second information content.

18. The label according to claim 17, wherein the first label area comprises an electronic display of a first display type and wherein the electronic display of the second label area is an electronic display of a second display type, and wherein the first display type and the second display type are different display types.

19. The label according to claim 18, wherein the first display type is one of an electroluminescent display, an electrophoretic display, a liquid crystal display, or a light emitting diode display, and wherein the second display type is one of an electroluminescent display, an electrophoretic display, a liquid crystal display, or a light emitting diode display.

20. The label according to claim 18 , wherein the first display type is an electrophoretic display and wherein the second display type is a light emitting diode display.

21. The label according to claim 17, wherein the first information content is printed or imprinted on the first label area.

22. The label according to claim 17, wherein the electronic display of the second label area comprises an organic light emitting diode display.

23. The label according to claim 17, wherein the electronic display of the second label area comprises a color display configured to visualize at least one of the second information content or an information background in at least two different colors.

24. The label according to claim 17, further comprising at least one of a device sensor or an ambient sensor, wherein the device sensor is configured to determine at least one of a position or orientation of at least one of a dose tracker or a piston rod of the injection device relative to a housing or body of the injection device and wherein the ambient sensor is configured to determine at least one of an ambient brightness, an acceleration, a temperature, or a variation of ambient air pressure.

25. The label according to claim 24, wherein the processor is operable to modify at least one of the first information content or the second information content depending on an electric signal received from at least one of the device sensor or the ambient sensor.

26. The label according to claim 24, wherein the device sensor comprises an electrode structure on the substrate configured to measure at least one of an electric capacitance, an electric field, or a magnetic field.

27. The label according to claim 26, wherein the electronic display comprises at least one electrically conductive grid electrode at least partially spatially overlapping with the electrode structure of the device sensor, and wherein the at least one electrically conductive grid electrode provides an electromagnetic shield for the electrode structure of the device sensor.

28. The label according to claim 17, wherein the processor comprises a power management operable to switch on and/or to switch off the electronic display.

29. The label according to claim 28, wherein the power management is configured to switch on, switch off, and/or dim the electronic display dependent on varying ambient conditions.

30. An injection device for setting and injecting of a dose of a medicament, the injection device comprising:
a housing configured to accommodate a medicament container;
a drive mechanism configured to withdraw or to expel the dose of the medicament from the medicament container and configured to inject the dose of the medicament into biological tissue; and
a label attached to the housing, wherein the label comprises:
a flexible substrate configured for attachment to the housing of the injection device,
a first label area on the substrate provided with a first information content, the first information content comprising at least one visual sign or character,
a second label area on the substrate non-overlapping with the first label area, wherein the second label area comprises an electronic display configured to display a second information content, and
a processor connected to the electronic display and configured to modify at least the second information content.

31. The injection device according to claim 30, further comprising the medicament container arranged inside the housing.

32. An adapter configured for a releasable attachment to a housing of an injection device, the adapter comprising:
a rigid body comprising an outside surface and a counter fastening feature configured to releasably mechanically engage with a fastening feature of the housing of the injection device; and
a label attached to the outside surface, wherein the label comprises:
a flexible substrate configured for attachment to the outside surface,
a first label area on the substrate provided with a first information content, the first information content comprising at least one visual sign or character,
a second label area on the substrate non-overlapping with the first label area , wherein the second label area comprises an electronic display configured to display a second information content, and
a processor connected to the electronic display and configured to modify at least the second information content.

33. The adapter according to claim 32, wherein the label further comprises at least one of a device sensor or an ambient sensor, wherein the device sensor is configured to determine at least one of a position or orientation of at least one of a dose tracker or a piston rod of the injection device relative to a housing or body of the injection device and wherein the ambient sensor is configured to determine at least one of an ambient brightness, an acceleration, a temperature, or a variation of ambient air pressure.

34. The adapter according to claim 33, wherein the processor is operable to modify at least one of the first information content or the second information content depending on an electric signal received from at least one of the device sensor or the ambient sensor.

35. The adapter according to claim 33, wherein the device sensor comprises an electrode structure on the substrate configured to measure at least one of an electric capacitance, an electric field, or a magnetic field.

36. The adapter according to claim 35, wherein the electronic display comprises at least one electrically conductive grid electrode at least partially spatially overlapping with the electrode structure of the device sensor, and wherein the at least one electrically conductive grid electrode provides an electromagnetic shield for the electrode structure of the device sensor.
